(19) United States
(12) Patent Application Publication
(10) Pub. No.: US 2015/0290231 A1
(43) Pub. Date: Oct. 15, 2015

(54) COMPOSITION FOR INHIBITING CELLULAR SENESCENCE COMPRISING QUERCETIN-3-O-beta-D-GLUCURONIDE

(71) Applicant: INDUSTRY-ACADEMIC COOPERATION FOUNDATION, YEUNGNAM UNIVERSITY, Gyeongsan-si (KR)

(72) Inventors: Jae-Ryong Kim, Daegu (KR); Jong-Keun Son, Daegu (KR); Hyo-Hyun Yang, Daegu (KR); Kyoung Hwangbo, Gyeongsan-si (KR)

(73) Assignee: Industry-Academic Cooperation Foundation, Yeungnam University , a foundation

(21) Appl. No.: 14/425,951

(22) PCT Filed: Sep. 5, 2013
(86) PCT No.: PCT/KR2013/008031

§ 371 Date: Mar. 4, 2015

(30) Foreign Application Priority Data
Sep. 6, 2012 (KR) 10-2012-0098811

(51) Int. Cl. A61K 31/7048; A61K 36/704

(52) U.S. Cl. CPC A61K 31/7048; A61K 36/704; A61K 2236/35

(57) ABSTRACT

The present invention relates to a composition for inhibiting cellular senescence comprising quercetin-3-O-β-D-glucuronide (Q3GA) as an active ingredient, and provides a composition for inhibiting cellular senescence of fibroblasts or umbilical vein endothelial cells induced by adriamycin or replicative senescence of fibroblasts or umbilical vein endothelial cells. Specifically, the quercetin-3-O-β-D-glucuronide (Q3GA) is characterized by being isolated from Polygoni avicularis herba extract. The composition may be usefully used for treating aging-related diseases, for example, skin aging, rheumatoid arthritis, osteoarthritis, hepatitis, chronically damaged skin tissue, arteriosclerosis, prostatic hyperplasia and liver cancer, by inhibiting a cellular senescence process of human fibroblasts and umbilical vein endothelial cells.

### TECHNICAL FIELD

The present invention relates to a composition for inhibiting cellular senescence comprising quercetin-3-O-β-D-glucuronide(Q3GA) as an active ingredient.

### BACKGROUND ART

Normal somatic cells reveal a finite number of cell proliferations due to cellular senescence. Cellular senescence is induced by diverse factors, such as telomere shortening due to DNA end replication problem (Collado et al., Cell. (2007) 130:223-233), altered activities of tumor suppressor genes and oncogenes, inflammation, oxidative stress, chemotherapeutic agents, and exposure of UV irradiation and ionizing radiation (Kuilman et al., Genes & Development. (2010) 24:2463-2479). Senescent cells show enlarged and flatten cell morphology, growth arrest, DNA damage foci in the nucleus, senescence-associated secretory phenotypes (SASP), and senescence-associated β-galactosidase (SA-β-gal) activity (Dimri et al., Proc Natl Acad Sci USA. (1995) 92:9363-9367; Rodier and Campisi, J Cell Biol. (2011) 192:547-556). Although diverse factors were known to induce cellular senescence, two tumor suppressor pathways, p53/p21 and Rb/p16, play a critical role in the regulation of cellular senescence (Campisi, Curr Opin Genet Dev. (2011) 21:107-112).

Accumulating evidence suggests that cellular senescence contributes to tissue and organismal aging, tissue repair and regeneration, and cancer progression and protection. In addition, cellular senescence is causally implicated in the pathogenesis of diverse age-related diseases, including cancer, atherosclerosis, skin aging, neurodegenerative disease, muscle atrophy, osteoporosis, and benign prostate hyperplasia. Recent evidence implies that inhibition of cellular senescence or removal of senescent cells in vivo can prevent or delay age-associated tissue dysfunction and extend healthspan. Telomerase null mice revealed premature aging phenotypes, restoration of telomerase in telomerase null mice decreased DNA damage signaling and rescued degenerative phenotypes across multiple organs (Jaskelioff et al., Nature. (2011) 469:102-106). Elimination of p16INK4a-positive senescence cells in the BubR1 progeroid mice delayed onset of age-related tissue phenotypes and attenuated progression of already established age-related disorders (Baker et al., Nature. (2011) 479:232-236). In mice, hepatic stellate cells are aged during hepatic fibrosis process, and it has been known that the aging of hepatic stellate cells inhibits excessive hepatic fibrosis. It has been known that too high p53 activity, without being properly controlled, accelerates senescence, but on the contrary, proper p53 activity inhibits senescence.

And, some study results about materials having cellular senescence inhibitory effect also reported. Drugs or single component such as vitamin C, N-acetylcysteine, NS398 and epifriedelanol inhibit the cellular senescence (Won-Sang et al., Nutrition Research and Practice. (2007) 1 :105-112; Kim et al., Mech Ageing Dev. (2008) 129:706-713; Yang et al., Planta Med. (2011) 77:441-449). And, it was reported that rapamycin in a mouse model and 4,4′-diaminodiphenylsulfone in Caenorhabditis elegans inhibit generation of aging-related diseases, and expand health-adjusted life expectancy (Harrison et al., Nature. (2009) 460:392-395; Cho et al., Proc Natl Acad Sci USA. (2010) 107:19326-19331).

Polygoni avicularis herba is also called knotgrass, and has antioxidant effect. It also known to have various effects such as: improving sperm mobility, which is reduced by electromagnetic wave exposure, in mouse model; recovering gum inflammation in human; inhibiting bile duct ligation-induced hepatic fibrosis; recovering acetaminophen-induced nephrotoxicity; and releasing vascular smooth muscle cells and thereby expanding blood vessels (Milan et al., Pak J Biol Sci. (2011) 14: 720-724; Sohn et al., Environ Toxicol Pharmacol. (2009) 27:225-230; Yin et al., J Ethnopharmacol. (2005) 99:113-117).

On the other hand, the present inventors disclosed a pharmaceutical composition for inhibiting aging comprising herb extract, which is at least one selected from the group consisting of Rhei rhizoma, Cirsii Radix, Plantaginis semen, Cinnamoni cortex, Cinnamoni cortex spissus, Euonimi lignum suberalatu, Salicis radicis cortex, Polygoni avicularis herba and Chaenomelis langenariae radix, as an active ingredient in Korean Patent Publication No. 10-2011-0041710, but there was no mention about quercetin-3-O-β-D-glucuronide (Q3GA) compound of the present disclosure.

### DISCLOSURE

### Technical Problem

The present disclosure is directed to providing a composition for inhibiting cellular senescence comprising quercetin-3-O-β-D-glucuronide (Q3GA) as an active ingredient.

The present disclosure is also directed to providing a pharmaceutical composition for inhibiting cellular senescence comprising quercetin-3-O-β-D-glucuronide (Q3GA) as an active ingredient, which may exert therapeutic effect for skin aging, rheumatoid arthritis, osteoarthritis, hepatitis, chronically damaged skin tissue, arterio sclerosis, prostatic hyperplasia, liver cancer and the like.

The present disclosure is also directed to providing a use of quercetin-3-O-β-D-glucuronide (Q3GA) for preparing the composition for inhibiting cellular senescence, or a method for inhibiting cellular senescence, which comprises a step of administrating therapeutically effective amount of quercetin-3-O-β-D-glucuronide (Q3 GA) into a subject.

### Technical Solution

As one general aspect, in the present disclosure, cellular senescence inhibitory effect of 12 kinds of single component, isolated and purified from Polygoni avicuiaris herba extract, in human fibroblasts and umbilical vein endothelial cells was examined. As a result, the present disclosure was completed by founding that, of them, quercetin-3-O-β-D-glucuronide (Q3GA) inhibits adriamycin-induced cellular senescence in human fibroblasts and umbilical vein endothelial cells, and also inhibits cellular senescence in replicative senescence-induced cells.

The present disclosure provides a composition for inhibiting cellular senescence comprising quercetin-3-O-β-D-glucuronide (Q3GA), represented by the following Chemical Formula 1, as an active ingredient. Specifically, the quercetin-3-O-β-D-glucuronide (Q3GA) may be isolated from Polygoni avicuiaris herba extract, and more specifically, the Polygoni avicularis herba extract may be prepared by adding butanol (n-BuOH) to a distilled water layer, which is fractionated after adding ethyl acetate (EtOAc) to a distilled water layer, which is fractionated after adding distilled water and hexane (n-hexane) to Polygoni avicularis herba methanol extract, and then fractionating thereof.

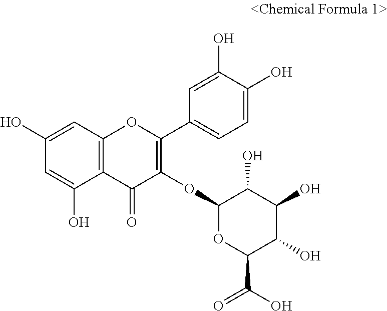 [SMILES: O=C(O)[C@H]1O[C@@H](Oc2c(-c3ccc(O)c(O)c3)oc3cc(O)cc(O)c3c2=O)[C@H](O)[C@@H](O)[C@@H]1O]

The quercetin-3-O-β-D-glucuronide (Q3GA) of Chemical Formula 1 may be isolated from natural materials, specifically, plants. It may be isolated from various organs, roots, stems, leaves, flowers and plant tissue culture extracts of natural, cross and variety plants. The most specifically, it may be isolated from Polygoni avicularis herba.

Specifically, the cellular senescence may be senescence or replicative senescence of fibroblasts or umbilical vein endothelial cells, and the senescence of fibroblasts or umbilical vein endothelial cells may be induced by adriamycin.

Further, the cellular senescence inhibitory effect may be determined by measuring inhibition of senescence-associated β-galactosidase (SA-β-gal) activity or inhibition of p53 expression.

Further, the composition of the present disclosure may be provided in various forms selected from a pharmaceutical composition or a functional food composition.

When the composition of the present disclosure is a pharmaceutical composition, the pharmaceutical composition may contains pharmaceutically acceptable carriers other than the quercetin-3-O-β-D-glucuronide (Q3GA), and the pharmaceutically acceptable carriers may be carriers generally used for formulating drugs, for example, lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methyl cellulose, methylhydroxy benzoate, propylhydroxy benzoate, talc, magnesium stearate, mineral oil and the like, but not limited thereto. Further, the pharmaceutical composition may further contain additives such as lubricants, humectants, sweetening agents, flavoring agents, emulsifiers, suspending agents and preservatives.

A method for administrating the pharmaceutical composition may be determined according to the degree of cellular senescence, and generally, it may be a local administration method. Further, therapeutically effective amount of the active ingredient in the pharmaceutical composition may differ from administration route, severity of disease, age, gender and body weight of a patient, and the like, and for example, daily dosage may be 0.01 to 1,000 mg/kg, specifically 0.1 to 1,000 mg/kg, more specifically 0.1 to 100 mg/kg. The administration may be made once a day or several times a day.

The pharmaceutical composition may be administered into mammals as a subject including rat, mouse, cattle and human through various routes. All administration routes may be employed, for example, oral, rectal, intravenous, intramuscular, subcutaneous, intrauterine, epidural or intracerebroventricular routes.

The pharmaceutical composition may be manufactured in a single-dose formulation or enclosed in a multiple-dose vial by formulating using pharmaceutically acceptable carriers and/or excipients. At this time, the formulation may be in the form of solutions, suspensions or emulsions, or elixirs, extracts, powders, granules, tablets, plaster, lotions or ointments.

On the other hand, the pharmaceutical composition may treat any one disease selected from the group consisting of skin aging, rheumatoid arthritis, osteoarthritis, hepatitis, chronically damaged skin tissue, arteriosclerosis, prostatic hyperplasia and liver cancer, but not limited thereto.

Further, when the present disclosure is a food composition, the kind of the food may not be particularly limited. Examples of foods, to which the quercetin-3-O-β-D-glucuronide (Q3GA) can be added, may include meats, sausages, breads, chocolates, candies, snacks, confectioneries, pizza, instant noodles, other noodles, gums, dairy products including ice cream, various soups, beverages, teas, drinks, alcoholic beverages and multi-vitamin preparations.

### Advantageous Effects

The present inventors confirmed that the quercetin-3-O-β-D-glucuronide (Q3GA) compound isolated from Polygoni avicularis herba inhibits adriamycin-induced cellular senescence, and also inhibits cellular senescence in replicative senescence-induced cells. It may be usefully used for treating aging-related diseases, for example, skin aging, rheumatoid arthritis, osteoarthritis, hepatitis, chronically damaged skin tissue, arteriosclerosis, prostatic hyperplasia and liver cancer, by inhibiting the cellular senescence process of human fibroblasts and umbilical vein endothelial cells.

### DESCRIPTION OF DRAWINGS

FIG. 1 represents the effect of quercetin-3-O-β-D-glucuronide (Q3GA) on adriamycin-induced cellular senescence in human dermal fibroblasts (HDFs). Cells treated with 500 nM adriamycin for 4 h were seeded at 500 cells/well in 96 well plates. After treatment with increasing concentrations of Q3GA, cells were incubated for 3 days and cellular senescence assessed by SA-β-gal activity staining. SA-β-gal activity staining (Left) and percentages of SA-β-gal positive cells in HDFs (Right). Representative SA-β-gal staining pictures of 3 independent experiments are shown. Values are means±SDs from 3 independent experiments measured in triplicates. C, control; D, dimethyl sulfoxide; N, 5 mM N-acetylcysteine; R, 500 nM rapamycin. *p<0.05 or **p<0.01 vs DMSO.

FIG. 2 represents the effect of quercetin-3-O-β-D-glucuronide (Q3GA) on adriamycin-induced cellular senescence in human umbilical vein endothelial cells (HUVECs). Cells treated with 500 nM adriamycin for 4 h were seeded at 1,000 cells/well in 96 well plates. After treatment with increasing concentrations of Q3GA, cells were incubated for 3 days and cellular senescence assessed by SA-β-gal activity staining. SA-β-gal activity staining (Left) and percentages of SA-β-gal positive cells in HUVECs (Right). Representative SA-β-gal staining pictures of 3 independent experiments are shown. Values are means±SDs from 3 independent experiments measured in triplicates. C, control; D, dimethyl sulfoxide; N, 5 mM N-acetylcysteine; R, 500 nM rapamycin. *p<0.05 or **p<0.01 vs DMSO.

FIG. 3 represents the effect of Q3GA on the expression levels of p53, pS6K, and p21 proteins in HDFs and HUVECs treated with adriamycin. Cells were treated with the indicated concentrations of Q3GA for 1 h prior to adriamycin treatment and incubated for 4 h. Proteins from cells were extracted and separated. The expression levels of each protein were analyzed by Western blotting. Representative data of 3 independent experiments are shown. NT, not treated with adrimycin; ADR, adriamcyin; C, control; D, dimethyl sulfoxide; N, 5 mM N-acetylcysteine; R, 500 nM rapamycin.

FIG. 4 represents the effect of Q3GA on intracellular ROS levels increased by adriamycin treatment in HDFs. Young cells (1.5×10⁵) treated with or without adrimcyin were seeded in 60 mm culture dishes and incubated for 24 h. Following treatment of cells with 10 g/mL of Q3GA, 0.5% DMSO, 5 mM NAC, or 500 nM rapamcyin for 3 days, cells were loaded with 250 M H₂DCFDA for 20 min. The DCF fluorescence intensity of each population of 10,000 cells was measured by flow cytometry. Representative data from three independent experiments are shown. Median fluorescence intensities were obtained and compared. Values are means±SDs from 3 independent experiments. NT, not treated with adrimycin; ADR, adriamcyin; C, control; D, dimethyl sulfoxide; N, 5 mM N-acetylcysteine; R, 500 nM rapamycin. *p<0.05 vs DMSO.

FIG. 5 represents the effect of Q3GA on intracellular ROS levels increased by adriamycin treatment in HUVECs. Young cells (1.5×10⁵) treated with or without adrimcyin were seeded in 60 mm culture dishes and incubated for 24 h. Following treatment of cells with 10 g/mL of Q3GA, 0.5% DMSO, 5 mM NAC, or 500 nM rapamcyin for 3 days, cells were loaded with 250 M H₂DCFDA for 20 min. The DCF fluorescence intensity of each population of 10,000 cells was measured by flow cytometry. Representative data from three independent experiments are shown. Median fluorescence intensities were obtained and compared. Values are means±SDs from 3 independent experiments. NT, not treated with adrimycin; ADR, adriamcyin; C, control; D, dimethyl sulfoxide; N, 5 mM N-acetylcysteine; R, 500 nM rapamycin. *p<0.05 vs DMSO.

FIG. 6 represents the effect of Q3GA on replicative senescence of HDFs. Old cells (3×10⁴/well) were seeded in 6 well plates and incubated with or without 10 g/mL of Q3GA for 3 days. Cellular senescence was assessed by SA-β-gal activity staining. Representative pictures of 3 independent experiments are shown. Percentages of SA-β-gal positive cells were measured. Values are means±SDs from 3 independent experiments. O, old cells; D, dimethyl sulfoxide; N, 5 mM N-acetylcysteine; R, 500 nM rapamycin. *p<0.05 or **p<0.05 vs DMSO.

FIG. 7 represents the effect of Q3GA on replicative senescence of HUVECs. Old cells (3×10⁴/well) were seeded in 6 well plates and incubated with or without 10 g/mL of Q3GA for 3 days. Cellular senescence was assessed by SA-β-gal activity staining. Representative pictures of 3 independent experiments are shown. Percentages of SA-β-gal positive cells were measured. Values are means±SDs from 3 independent experiments. O, old cells; D, dimethyl sulfoxide; N, 5 mM N-acetylcysteine; R, 500 nM rapamycin. *p<0.05 or **p<0.05 vs DMSO.

The file of this patent contains at least one drawing executed in color. Copies of this patent with color drawing(s) will be provided by the Office upon request and payment of the necessary fee.

### BEST MODE

The examples and experiments will now be described. The following examples and experiments are for illustrative purposes only and not intended to limit the scope of the present disclosure.

### Example 1

### Isolation and Structure Identification of quercetin-3-O-β-D-glucuronide (Q3GA)

1. Isolation of quercetin-3-O-β-D-glucuronide (Q3GA)

The dried aerial parts of P. aviculare L. (9 kg) were extracted with 70%, 90% and 100% MeOH by reflux for 24 hr, successively, and the combined MeOH solution was evaporated to dryness (1.4 kg). The dried MeOH extract (1.4 kg) was resuspended in 1.5 L distilled water and the solution was partitioned with n-hexane (1.5 L×3), ethyl acetate (EtOAc, 1.5 L×3), and n-butanol (n-BuOH, 1.5 L×3), successively. After drying, four solvent extracts of n-hexane (120 g), EtOAc (65 g), n-BuOH (140 g), and H₂O (800 g) were obtained.

The n-BuOH extract (10 g) was loaded on a sephadex LH-20 column (4×90 cm), and the column was eluted with MeOH. The eluent was combined on the basis of TLC, giving 17 fractions (PAB 1-17). Of these fractions, PAB 12 gave quercetin-3-O-β-D-glucuronide (Q3GA, PAC11, 15 mg) by a reverse-phase column chromatography (4×50 cm) with MeOH—H₂O (gradient from 15% to 35%). The Q3GA was dissolved in dimethyl sulfoxide, and then treated to cells.

2. Structure Identification of quercetin-3-O-β-D-glucuronide

Chemical structures of the quercetin-3-O-β-D-glucuronide isolated from Polygoni avicularis herba extract was identified by comparing the following spectroscopic analysis data with reference (J. agric. Food Chem. 1998, 46, 4898-4903) (Chemical Formula 1). Spectroscopic analysis data was as follows.

Brown powder C₂₂H₂₀O₁₂ m.p. 190-192° C. ¹H-NMR (250 MHz, Methanol-d4) 7.92˜7.76 (2H, m, H-2′, 6′), 6.84 (1H, d, J=9.0 Hz, H-5′), 6.38 (1H, d, J=2.0 Hz, H-8), 6.19 (1H, d, J=2.0 Hz, H-6), 5.33 (1H, d, J=7.4 Hz, H-1″), 3.77 (1H, d, J=9.5 Hz, H-5″), 3.42-3.62 (3H, m, H-2″, 3″, 4″) ¹³C-NMR (62.9 MHz, Methanol-d4) 179.2 (C═O), 172.3 (C-6″), 166.0 (C-7), 163.0 (C-5), 159.0 (C-2), 158.4 (C-9), 150.0 (C-4′), 146.0 (C-3′), 135.4 (C-3), 123.5 (C-6′), 122.8 (C-1′), 117.2 (C-5′), 116.0 (C-2′), 105.6 (C-10), 104.3 (C-1″), 99.9 (C-6), 94.8 (C-8), 77.6 (C-3″), 77.1 (C-5″), 75.4 (C-2″), 72.9 (C-4″) Positive FABMS m/z 460.1 [M-OH+H]+ [α]_{D}²⁵−24.5° (c 0.66 MeOH).

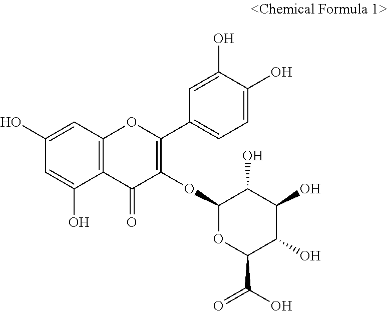 [SMILES: O=C(O)[C@H]1O[C@@H](Oc2c(-c3ccc(O)c(O)c3)oc3cc(O)cc(O)c3c2=O)[C@H](O)[C@@H](O)[C@@H]1O]

### Example 2

### Examination of Cellular Senescence Inhibitory Effect of quercetin-3-O-β-D-glucuronide (Q3GA) in Human Fibroblasts and Umbilical Vein Endothelial Cells

1. Materials

Human dermal fibroblasts (HDFs) and human umbilical vein endothelial cells (HUVECs) were obtained from Lonza (Walkersvill, Md., USA). Dulbecco's Modified Eagle medium (DMEM), fetal bovine serum (FBS), and penicillin-streptomycin solution were from WelGene (Daegu, Republic of Korea). Endothelial cell growth medium-2(EGM-2) were Lonza (Walkersvill, Md., USA). Antibodies against p53 and p21 were from Santa Cruz Biotech. Inc. (Santa Cruz, Calif., USA) and an antibody against phosphorylated S6 kinase from Cell Signaling Technology Inc. (Beverly, Mass., USA). A rabbit polyclonal antibody against glyceraldehydes 3-phosphate dehydrogenase (GAPDH) was kindly donated by Dr. K. S. Kwon (KRIBB, Daejeon, Republic of Korea).

2. Cell Culture

HDFs in DMEM with 10% FBS and 1% antibiotics (penicillin 10,000 unit/mL and streptomycin 10,000 mg/mL) were seeded at 1×10⁵ cells per 100 mm culture plate and incubated at 37° C. in 5% CO₂ humidified air. When subcultures reached 80-90% confluence, serial passaging was performed by trypsinization. HUVECs in EGM-2 media were cultured under the same conditions. The number of population doublings (PDs) was monitored for further experiments. PD was calculated using the geometric equation: PD=log 2F/log 2I (F, final cell number; I, initial cell number). HDFs in PD<35 and HUVECs in PD<30 were used for adriamycin-induced cellular senescence. HDFs in PD>75 and HUVECs in PD>50 were used as old cells under replicative senescence.

3. Induction of Cellular Senescence by Adriamycin Treatment

HDFs in DMEM media or HUVECs in EGM-2 media were plated at 1.5×10⁵ cells per 100 mm culture plate. After incubation at 37° C. in a CO₂ incubator for 3 days, cells medium remove. Following rinsing 3 times with DMEM containing 1% antibiotics, HDFs in DMEM containing 10% FBS and 1% antibiotics and HUVECs in EGM-2 were incubated in a 5% CO₂ incubator for 4 days. Adriamycin-induced cellular senescence was confirmed by senescence-associated β-galactosidase (SA-β-gal) activity staining.

4. Examination of Effect of Single Compound on Adriamycin-induced Cellular Senescence

Whether single compound was effective on adriamycin-induced cellular senescence or not was examined. The cells treated with the adriamycin for 4 hours were separated from the culture dish with trypsin-EDTA. The fibroblasts were made to the cell concentration of 5,000 cells/ml in DMEM medium containing 10% fetal bovine serum and 1% antibiotics, and the umbilical vein endothelial cells were made to the cell concentration of 10,000 cells/ml in EGM-2 medium, and then 100 ml of them were divided into each well of a 96-well cell culture plate. Finally, 500 cells of the fibroblasts and 1,000 cells of the umbilical vein endothelial cells were divided into each well, respectively, and then cultured at 37° C. in a 5% CO₂ incubator for 1 day. 100 ml of DMEM medium containing 10% fetal bovine serum and 1% antibiotics and EGM-2 medium were further added into each well, respectively, and the Polygoni avicularis herba single compound was treated thereto to the concentration of 10 mg/ml. Dimethyl sulfoxide as a negative control group, and N-acetylcysteine 5 mM and rapamycin 500 nM as positive control groups were added to the cells, respectively. Then, the cells were cultured at 37° C. in a 5% CO₂ incubator for 3 days. The degree of cell growth was examined by MTT assay, and the degree of cellular senescence was examined by senescence-associated β-galactosidase (SA-β-gal) activity staining assay.

5. MTT Assay

The degree of cell growth was measured by 3-(4,5-dimethylthiazol-2yl)-2,5-diphenyltetrazolium bromide (MTT) assay. 0.1% MTT solution 50 μl was added to each well of a 96-well culture plate, and reacted at 37° C. in a 5% CO₂ incubator for 3 hours. The medium and the MTT solution were removed, and then dimethyl sulfoxide 100 μl was added thereto so as to dissolve formed crystals. Absorbance at 550 nm was measured by using a microplate reader.

6. Senescence-Associated β-Galactosidase (SA-β-gal) Activity Staining

The effect of single component on cellular senescence was examined by SA-β-gal activity staining. Each single component was treated in a 24-well culture plate or a 12-well culture plate for 3 days, and then the cells were washed with phosphate buffer. After fixing the cells with 3.7% paraformaldehyde, 250 μl of SA-β-gal staining solution [40 mM citric acid/phosphate, pH 5.8, 5 mM potassium ferrocyanide, 5 mM potassium ferricyanide, 150 mM NaCl, 2 mM MgCl₂, X-gal 1 mg/ml] for the 24-well and 500 μl of the SA-β-gal staining solution for the 12-well were added to each well, respectively. The plates were wrapped with aluminum foil, and then reacted at 37° C. for 16 hours to 18 hours. The cells were washed two times with phosphate buffer (PBS), and then stained with 1% eosin solution for 1 min. The cells were washed two times with phosphate buffer, and then the cells stained blue were observed with an optical microscope. The degree of SA-β-gal activity was measured by counting the number of the cells, whose cytosols were stained blue, out of the total of about 50 to 100 cells and displayed as percentage (%).

7. Cell Protein Extraction

Each cells were divided into a 60 mm culture dish to the cell number of 1×10⁵, and then cultured at 37° C. in a 5% CO₂ incubator. The cells were washed two times with DMEM medium containing antibiotics, and the fractions of Polygoni avicularis herba extract and the compounds were pretreated thereto for 1 hour by concentration, followed by treating adriamycin 500 nM for 4 hours. The medium was removed, and then the cells were washed one time with phosphate buffer. Cell lysis solution [25 mM Tris-HCl (pH 7.6), 150 mM NaCl, 1% Tryton X-100, 0.5% sodium deoxycholate, 0.1% SDS, 1 mM Sodium vanadate, 5 mM NaF, protease inhibitor or 1 mM PMSF] 50 μl was added thereto. Entire solution and cells were collected by using a cell lifter, and then transferred to a microcentrifuge tube. The tube was reacted on ice for 30 min while vortexing the solution every 10 min. The tube was centrifuged at 12,000×g for 10 min, and then supernatant was transferred to a new tube. The amount of the protein in the solution was quantified by bicinchoninic acid (BCA) method (Pierce Biotechnology Inc., Rockford Ill., USA) by using bovine serum albumin as a standard protein.

8. Western Blot Analysis

Protein (30 μg) was separated by being electrophoresed through a 10% SDS-polyacrylamide gel. The protein was transferred to a nitrocellulose membrane, and then reacted in Tween-20-Tris buffered saline (TTBS) containing 5% dry whole milk for 1 hour. The nitrocellulose membrane was reacted with 5% dry whole milk-TTBS solution containing primary antibody against p53 or p21 overnight. The membrane was washed three times with TTBS solution, and then reacted with a horseradish peroxidase-conjugated secondary antibody for 3 hours. The membrane was washed five times with TTBS for every 5 min, and then the amounts of p53, p21 and pS6 were measured by using an enhanced chemiluminescence solution. The amount of the specific protein reacted with each antibody was measured by using a LAS-3000 imaging system (Fujifilm Corp., Stanford, Conn., USA). Whether the same amount of the protein was used in each experiment was confirmed by using glyceraldehyde-3-phosphate dehydrogenase (GAPDH) antibody.

9. Measurement of Intracellular Reactive Oxygen Species (ROS) Concentration

Each cells were divided into a 100 mm culture dish to the cell number of 1.5×10⁵, and then cultured at 37° C. in a 5% CO₂ incubator for 3 days. The cells were washed two times with DMEM medium containing antibiotics, and then adriamycin 500 nM was treated thereto for 4 hours. The cells were separated by treating trypsin-EDTA solution (2.5%), and then divided again into a 60 mm culture dish to the cell number of 1×10⁵ followed by culturing at 37° C. in a 5% CO₂ incubator. The medium was replaced, and then quercetin-3-O-β-D-glucuronide 10 μg/ml was treated to the cells. Dimethyl sulfoxide as a negative control group and N-acetylcysteine 5 mM and rapamycin 500 nM as positive control groups were added to the cells. The cells were cultured at 37° C. in a 5% CO₂ incubator for 3 days, washed two times with DMEM medium containing antibiotics, and then treated with H₂DCFDA 250 μM for 20 mM. The cells were washed two times with phosphate buffer, and separated by treating trypsin-EDTA solution (2.5%) followed by transferring to a microcentrifuge tube. The tube was centrifuged at 12,000×g for 10 min, and then supernatant was discarded. The cells were washed with 2% fetal bovine serum-containing phosphate buffer 1 ml, and then centrifuged again at 12,000×g for 10 min. The cells were washed two times as described above, and then 1% paraformaldehyde 1 ml was added thereto. ROS was measured by using BD FACS Canto II flow cytometry (BD Biosciences, San Jose, Calif.).

10. Statistical Analysis

3 sets of each experiment were repeated three times or more, and then mean and standard deviation were calculated. Statistical significance was analyzed using Student t-test, and p value less than 0.05 (p<0.05) was considered as significant.

11. Result

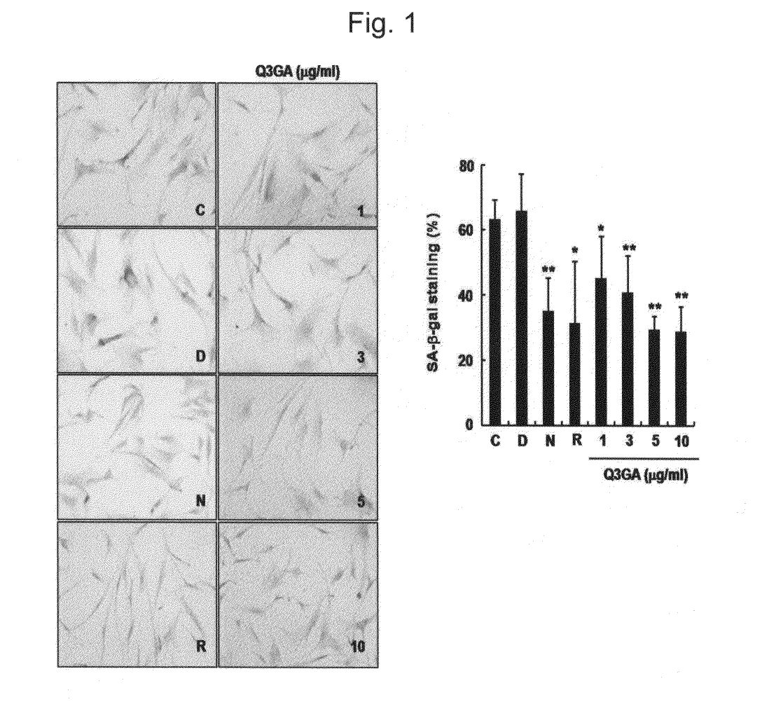

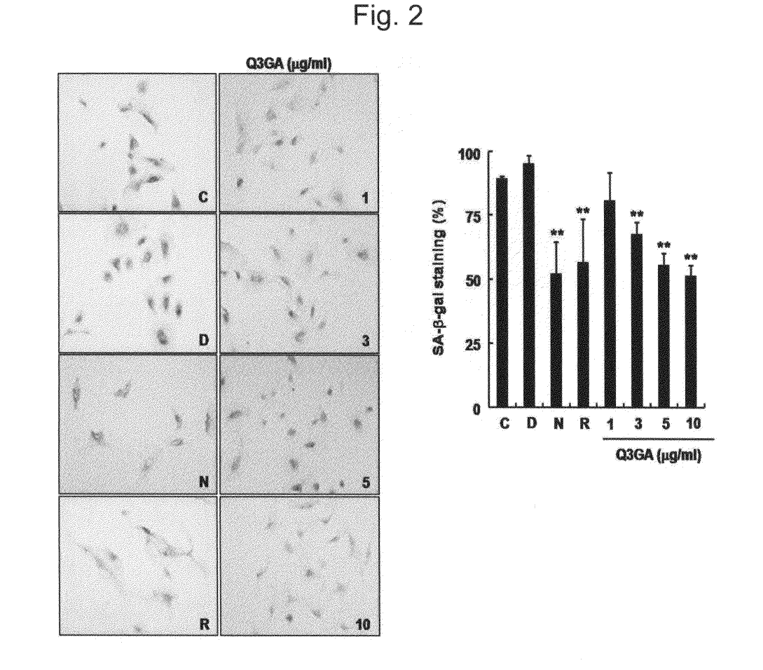

At first, we measured whether Q3GA isolated from P. aviculare showed cytotoxicity in HDFs and HUVECs. As a result, No cytotoxicity was observed at 10 g/mL of the compound. And then we investigated whether Q3GA would have inhibitory effect on cellular senescence in HDFs and HUVECs treated with adriamycin by observing activity staining of SA-β-gal, well known as a cellular senescence marker. And, it was compared with N-acetylcysteine (NAC) and rapamycin, reported to have the cellular senescence inhibitory effect. First of all, Q3GA 10 μg/ml was treated to the adriamycin-treated cells, and 3 day later, the degree of senescence was compared by SA-β-gal activity staining. As a result, in both of HDFs and HUVECs, the increased SA-β-gal activity staining induced by the adriamycin treatment was reduced by Q3GA treatment (FIG. 1 and FIG. 2). Further, whether the cellular senescence inhibitory effect of Q3GA is concentration-dependent or not was examined. As a result, it was confirmed that the increased SA-β-gal activity induced by adriamycin was reduced as Q3GA concentration was increased (FIG. 1 and FIG. 2).

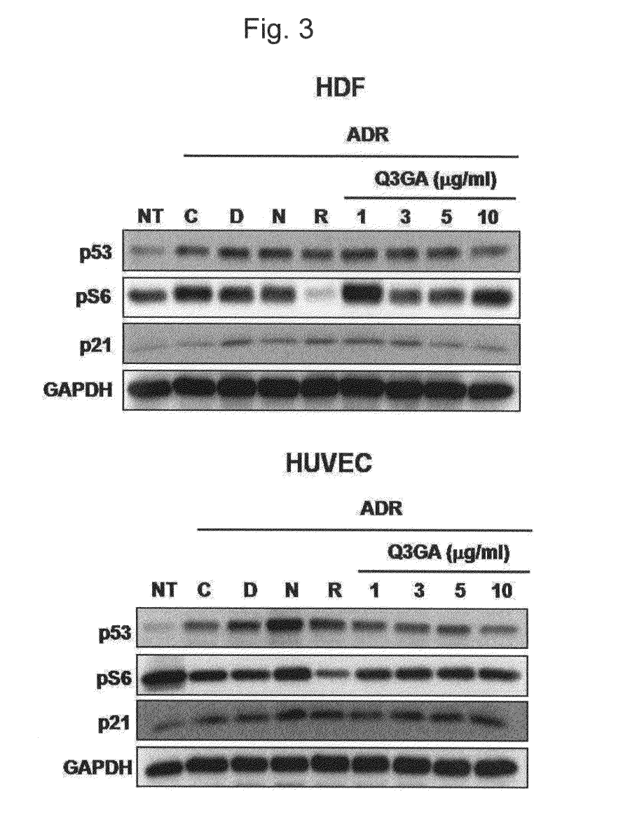

In addition, the proteins p53 and p21 are well-known markers increased in cellular senescence by adriamycin treatment. Therefore, we measured whether Q3GA decreases the expression levels of p53 and p21 in adriamycin-treated cells by western blot. As a result, Q3GA reduced the levels of p53 and p21 proteins increased by adriamycin treatment in both HDFs and HUVECs in a concentration-dependent manner (FIG. 3).

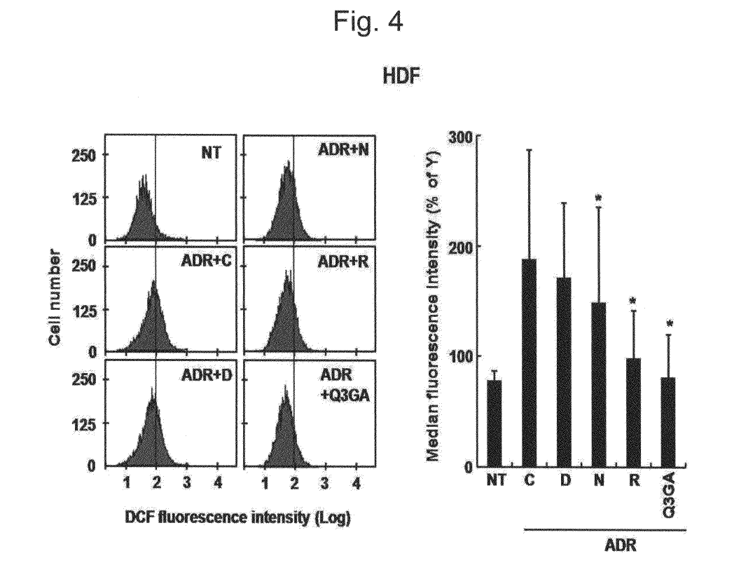

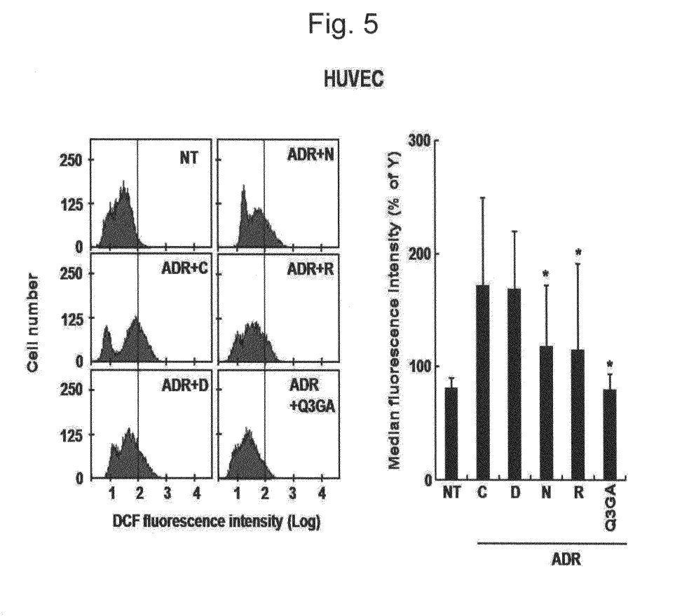

Since intracellular ROS levels are known to increase in cells during adriamycin-induced cellular senescence, we evaluated whether Q3GA would reduce ROS increased by adriamycin treatment in HDFs and HUVECs by measuring intracellular DCF fluorescence intensity. As a result, Q3GA decreased intracellular ROS levels augmented by adriamycin treatment (FIG. 4 and FIG. 5). In conclusion, we confirmed that Q3GA, purified from P. aviculare, has an inhibitory effect on cellular senescence induced by adriamycin in HDFs and HUVECs.

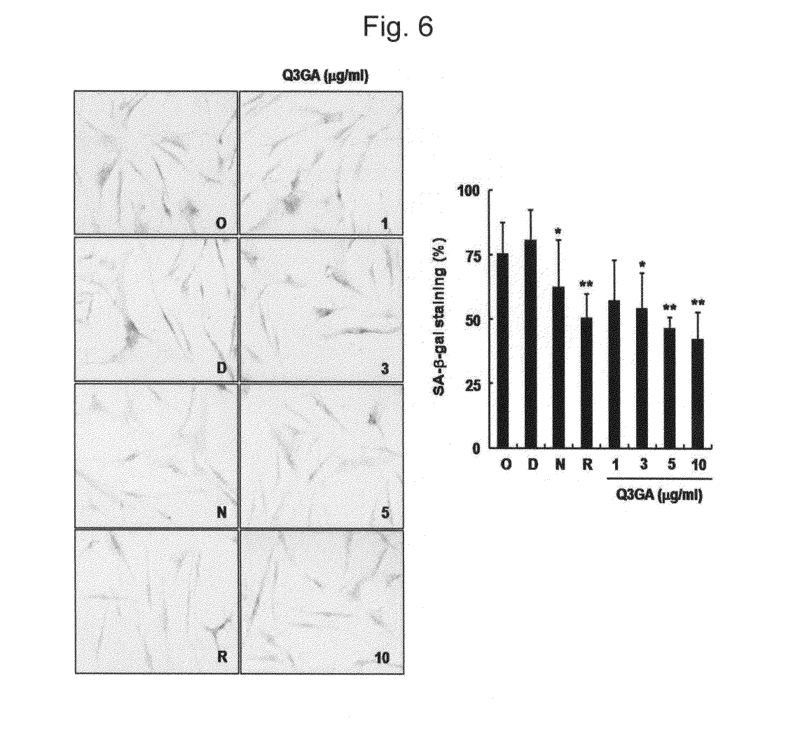

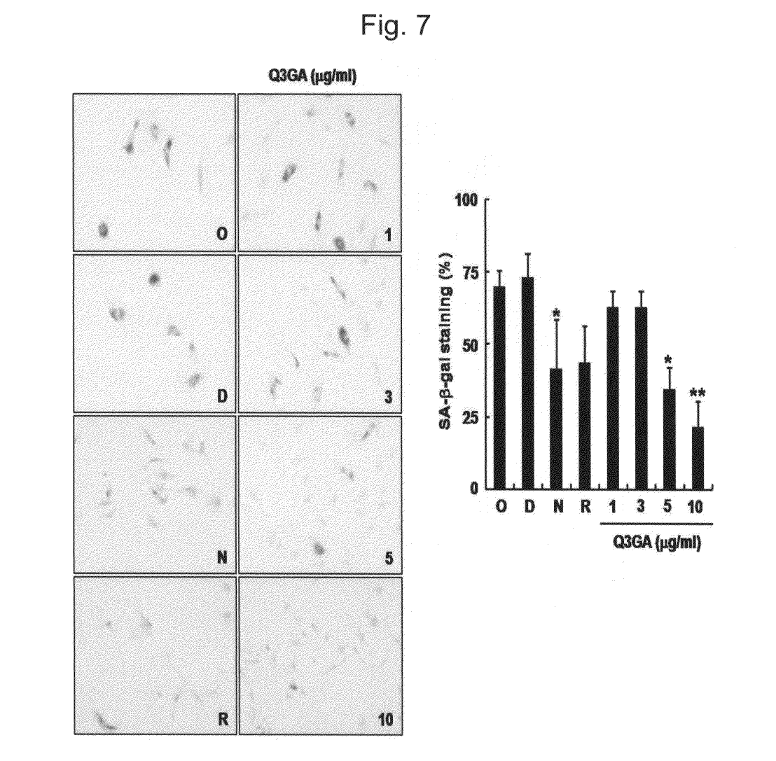

Meanwhile, Since Q3GA inhibited adriamycin-induced cellular senescence in HDFs and HUVECs, we elucidated whether Q3GA also reverse replicative senescence in both types of cells. In HDFs and HUVECs, the replicative senescence was induced by subculture. And, we examined the SA-β-gal activity in old cells while increasing the concentration of Q3GA. As a result, Q3GA decreased SA-β-gal activity in HDFs and HUVECs under replicative senescence in a concentration-dependent manner (FIG. 6 and FIG. 7), suggesting that Q3GA might be able to rescue replicative senescence in HDFs and HUVECs, in addition to adriamycin-induced cellular senescence.

Our data suggested that Q3GA repressed premature senescence induced by adriamycin treatment as well as replicative senescence which was confirmed by SA-β-gal activity and the levels of p53 and p21 proteins.

In conclusion, we found that Q3GA, purified from P. aviculare, has an inhibitory effect on cellular senescence in HDFs and HUVECs. This compound may be a promising candidate for developing dietary supplements or cosmetics to modulate tissue aging or aging-associated diseases.

Those skilled in the art will appreciate that the conceptions and specific embodiments disclosed in the foregoing description may be readily utilized as a basis for modifying or designing other embodiments for carrying out the same purposes of the present disclosure. Those skilled in the art will also appreciate that such equivalent embodiments do not depart from the spirit and scope of the disclosure as set forth in the appended claims.

1.-11. (canceled)

12. A method for inhibiting cellular senescence comprising a step of administrating therapeutically effective amount of quercetin-3-O-β-D-glucuronide (Q3GA) represented by the following Chemical Formula 1 into a subject in need thereof.

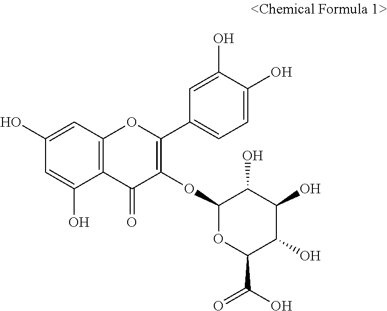 [SMILES: O=C(O)[C@H]1O[C@@H](Oc2c(-c3ccc(O)c(O)c3)oc3cc(O)cc(O)c3c2=O)[C@H](O)[C@@H](O)[C@@H]1O]

13. The method for inhibiting cellular senescence according to claim 12, wherein the quercetin-3-O-β-D-glucuronide (Q3GA) is isolated from a Polygoni avicularis herba extract.

14. The method for inhibiting cellular senescence according to claim 13, wherein the Polygoni avicularis herba extract is prepared by adding butanol (n-BuOH) to a distilled water layer, which is fractionated after adding ethyl acetate (EtOAc) to a distilled water layer, which is fractionated after adding distilled water and hexane (n-hexane) to Polygoni avicularis herba methanol extract, and then fractionating thereof.

15. The method for inhibiting cellular senescence according to claim 12, wherein the cellular senescence is senescence or replicative senescence of fibroblasts or umbilical vein endothelial cells.

16. The method for inhibiting cellular senescence according to claim 15, wherein the senescence of fibroblasts or umbilical vein endothelial cells is induced by adriamycin.

17. The method for inhibiting cellular senescence according to claim 12, wherein the effect of inhibiting cellular senescence is determined by measuring inhibition of senescence-associated β-galactosidase (SA-β-gal) activity or inhibition of p53 expression.
